(19) United States
(12) Patent Application Publication
(10) Pub. No.: US 2021/0121581 A1
(43) Pub. Date: Apr. 29, 2021

(54) METHODS AND COMPOSITIONS FOR TREATING TUMOR CELLS

(71) Applicant: Stitch Bio, LLC, Waltham, MA (US)

(72) Inventor: Anthony P. Shuber, Northbridge, MA (US)

(21) Appl. No.: 16/818,229

(22) Filed: Mar. 13, 2020

Related U.S. Application Data
(60) Provisional application No. 62/927,235, filed on Oct. 29, 2019.

(51) Int. Cl. A61K 48/00; C12N 15/113; C12N 9/22; C12Q 1/6886

(52) U.S. Cl. CPC A61K 48/0066; A61K 48/0058; C12N 15/113; C12Q 2600/156; C12Q 1/6886; C12N 2310/20; C12N 2800/80; C12N 9/22

(57) ABSTRACT

The disclosure provides methods and compositions that employ gene editing for the treatment of cancer. Gene editing systems specifically target tumor DNA to introduce an expression cassette with a coding sequence that is expressed by tumor cells as a neoantigen that mark the tumor cells for cell death.

### TECHNICAL FIELD

The disclosure relates to methods and compositions for treating tumor cells.

### BACKGROUND

Numerous attempts have been made to utilize the immune system for treatment and monitoring of disease. For example, in immuno-oncology several groups have attempted to induce neo-antigen expression in cancer cells to trigger a primary immune response directed against the cancer cells. In general, cancer cells evade the immune system by several mechanisms. For example, it is thought that down-regulation of major histocompatibility class antigens and lack of co-stimulatory signals for antigen production allow tumors to go undetected in early stages of cancer. Later, tumors may express immunosuppressive gene products that further reduce the ability of the immune system to act against the tumor cells.

Further, tumors take advantage of checkpoint proteins which inhibit the immune system from attacking “normal” cells. Thus, a current approach in immune-oncology is to create so-called checkpoint inhibitors that release the inhibition on T-cells, allowing those T-cells to kill tumor cells. Other approaches have used mutation-derived neoantigens to distinguish normal from tumor cells. In that approach, antibodies are used to increase the virulence of the immune system by increasing T-cell cytotoxicity targeted toward the neoantigens. These approaches have met with good initial success but not universal applicability due to the complexity of the immune response and the heterogeneity of presenting antigens in tumors.

### SUMMARY

The invention uses genome editing to selectively target tumor cell genomes for insertion of coding sequences that are then expressed by the tumor cells as neoantigens. A genome-editing tool such as a Cas endonuclease, or nucleic acid encoding the Cas endonuclease, is delivered to a patient along with the neoantigen coding sequence, which may be provided in an expression cassette. The genome editing tools selectively target a tumor genome by virtue of being designed to act on sequences found specifically in the tumor genome and not also in corresponding portions of matched normal sequences from the same patient. In the tumor cells, the genome editing tools target and cleave the tumor-specific sequences, resulting in insertion and integration of the exogenous coding sequences, e.g., by homology-directed end repair, into the tumor genome. The exogenous coding sequences may be provided as an expression cassette with regulatory sequences such as promoters or transcription factor binding sites that induce expression of those coding sequences as cell-surface proteins on the tumor cells that function as neoantigens. The expression of neoantigens via the invention results in expression of unmasked antigens that can be used to mark the tumor cells for death by, for example, the immune system or an antibody-drug-conjugate.

To kill the tumor cells, an immune response may be triggered simply by the expression of the neoantigens, by “training” the immune system with vaccination, or by targeting expressed neoantigens with drugs, such as antibody-drug conjugates, that target and destroy cells displaying the targeted antigen.

Accordingly, the invention provides methods and compositions for treating cancer in a subject. The invention relies on the expression of cell surface proteins on the surface of tumor cells to treat cancer in a subject. The invention uses gene editing such as Cas endonuclease to induce expression of cell surface proteins on tumor cells. Methods of the invention include introducing gene editing reagents as well as expression cassettes encoding at least a segment of a cell surface protein. The gene editing reagents may be delivered as active proteins, e.g., a ribonucleoprotein (RNP) that includes a Cas endonuclease complexed with a guide RNA, or as nucleic acid encoding the active gene editing reagents—e.g., as a second expression cassette encoding the Cas endonuclease and one or more guide RNAs. The gene editing tools insert the exogenous coding sequences into tumor-specific genomic material of tumor cells, thereby inducing expression of a cell surface protein on the tumor cells.

Methods may include identifying sequences found specifically in a tumor genome and not also in corresponding portions of matched normal sequences from the same patient and designing the gene editing tools (e.g., the guide RNAs) to bind to and act on those tumor-specific genomic sequences. Identifying tumor-specific sequences may include obtaining a patient sample and analyzing tumor DNA sequences from the sample to identify sequences that are in the tumor DNA but not also present in matched-normal DNA from the patient. For example, patient samples may be obtained that include tumor and non-tumor cells from any suitable source including germline or somatic sources. Sequencing may be performed, e.g., using next-generation sequencing instruments, and resulting tumor sequences may be compared and matched to corresponding sequences from non-tumor cells, the “matched normal” sequences. Sequences appearing exclusively in the tumor genome may thus be identified as the targets suitable for targeting with the gene editing systems. Delivering the exogenous coding sequences into cells with genome editing tools that only integrate those coding sequences into targets exclusive to tumor genomes, and allowing those coding sequences to be expressed by the tumor cells as cell-surface proteins allows the tumor cells to exhibit a novel antigen that can be targeted for cell death.

Methods of the invention include using a gene-editing system to induce expression of a cell surface protein in a tumor cell to thereby provide an antigen useful to target the tumor cell for dell death. The gene editing system delivered to the subject may include at least one Cas endonuclease or a nucleic acid encoding the Cas endonuclease. In some embodiments, the cell surface protein is an exogenous antigen and the Cas endonucleases include one or more guide RNAs that target delivery of the coding sequence for the exogenous antigen to a predetermined site in the tumor genome. The predetermined site may include, for example, a genomic safe harbor. The gene editing system may include at least a ribonucleoprotein (RNP) that includes a Cas endonuclease and a guide RNA (gRNA) that binds the RNP to a predetermined site within the tumor-specific genomic material and introduces the expression cassette into the tumor-specific genomic material. The expression cassette may also introduce a promoter or a transcription factor binding site to increase transcription of the coding sequence, e.g., the cell surface protein. The nucleic acid sequence of the promoter or the transcription biding site may be included along with the nucleic acid sequence of an antigen as an expression cassette.

Methods of the invention may also include modulating the immune system of a subject. For example, the immune system may be primed to exhibit a response against the antigen by administering the antigen or an epitope thereof. Preferably, the antigen or peptide is recognized by autologous T cells. By introducing the antigen to subject, the immune system responds to the presence of the antigen and begins to attack tumor cells expressing the antigen, thus treating cancer in the subject.

In certain aspects, the disclosure provides a method of treating a tumor cell. The method includes introducing, into a subject, a gene editing system and an expression cassette including a coding sequence encoding at least a segment of a cell surface protein. The gene editing system integrates the expression cassette into a genome of a tumor cell in the subject, thereby causing the tumor cell to express the coding sequence as a neoantigen. Preferably, the neoantigen marks the tumor cell for destruction by an immune response of the subject or an antibody-drug-conjugate. The gene editing system may include a targeting sequence that binds specifically to a target in the genome of the tumor cell. Preferably the target is not found in matched normal sequences from healthy, non-tumor cells of the subject. In some embodiments, the method includes delivering the neoantigen to the subject prior to the introducing step to thereby prime an immune system of the subject.

In certain embodiments, the gene editing system includes a ribonucleoprotein (RNP) that comprises a Cas endonuclease and a guide RNA, i.e., in which the guide RNA includes the targeting sequence. In other embodiments, the gene editing system includes at least one transcription activator-like effector nuclease (TALEN) with a primary amino acid sequence that confers target specificity on the TALEN to a target in the genome of the tumor cell in the subject.

The method may optionally include, prior to the introducing step, obtaining tumor DNA from the subject and analyzing the tumor DNA (e.g., by sequencing or probe hybridization assays) to identify a target in the tumor DNA that is not found in matched normal sequences from healthy, non-tumor cells of the subject. Embodiments may include sequencing matched, normal DNA from the healthy, non-tumor cells of the subject to thereby obtain tumor sequences and matched normal sequences; aligning the tumor sequences to the matched normal sequences; and identifying the target as a section of the tumor sequence that does not have an exact match in the matched normal sequences.

The method may further include obtaining or synthesizing one or more guide RNAs with targeting portions that are complementary to the target in the tumor DNA when the target in the tumor DNA is adjacent a protospacer adjacent motif in the tumor DNA.

In certain embodiments, the expression cassette further comprises a promoter operably linked to the coding sequence. In some embodiments, the neoantigen is recognized by a receptor on a T cell in the subject. The method may include administering, to the subject, an antibody-drug-conjugate (ADC) comprising an antibody that specifically binds the neoantigen. The ADC includes the antibody conjugated to a cytotoxic drug that kills the tumor cell.

The method may include analyzing a sample from the subject to identify a target in and specific to the genome of the tumor cell in the subject; obtaining guide RNA that hybridizing the target; introducing the guide RNA to a Cas endonuclease that includes a nuclear localization signal to form a ribonucleoprotein (RNP); and packaging the RNP and the expression cassette in one more lipid particles for delivery.

In other aspects, the disclosure provides a composition that includes a gene editing system—or nucleic acid encoding the gene editing system—and an expression cassette. The gene editing system includes a targeting sequence that binds specifically to a target in a tumor genome and the expression cassette includes a coding sequence encoding at least a segment of a cell surface protein. Preferably, the target in the tumor genome is not found in a genome from healthy, non-tumor cells of a subject with the tumor. When the composition is delivered to a subject, the gene editing system causes integration of the expression cassette into the tumor genome at the target. The integration results in expression of the coding sequence as an antigen on a tumor cell that includes the tumor genome.

In certain embodiments, the gene editing system includes a Cas endonuclease and a guide RNA that includes the targeting sequence, the Cas endonuclease and guide RNA being complexed as a ribonucleoprotein (RNP). The RNP, the expression cassette, or both may be packaged in one or more lipid particles for delivery, such as solid lipid nanoparticles or liposomes. For example, the composition may include at least dozens, or several hundred, or several thousand of the solid lipid nanoparticles packaging at least a corresponding number of the RNP and the expression cassette. The solid lipid nanoparticles may be packaged in a vessel or container such as a blood collection tube or a microcentrifuge tube. For example, in some embodiments, the container comprises a microcentrifuge tube. The solid lipid nanoparticles may be provided as an aqueous suspension in one or more such containers (e.g., with all tubes on optionally on dry ice in a Styrofoam container).

In some embodiments, the composition includes the nucleic acid encoding the gene editing system, e.g., as a plasmid or expression cassette. The expression cassette and the nucleic acid encoding the gene editing system may be provided in an aqueous solution.

In related embodiments, the disclosure provides a kit that includes any of the foregoing compositions in one or more suitable containers, the kit optionally including, in a separate container, a dose of the antigen that may be delivered to the subject to prime an immune system of the subject. The kit may further include, in a separate container, an antibody-drug-conjugate (ADC) comprising an antibody that specifically binds the neoantigen. The ADC may include the antibody conjugated to a cytotoxic drug that kills the tumor cell.

The various methods, compositions, and kits of the disclosure are useful for inducing expression of a cell surface protein on a tumor cell in a subject. Compositions preferably includes a gene editing system—or nucleic acid encoding the gene editing system—and nucleic acid encoding at least a segment of a cell surface protein. The composition may include the gene editing system as a Cas endonuclease complexed with a guide RNA that specifically hybridizes to targets in a tumor genome. The Cas endonuclease and guide RNA may be present as a ribonucleoprotein (RNP). The nucleic acid encoding at least a segment of a cell surface protein may be an expression cassette for an exogenous coding sequence with one or more of a promoter and a transcription factor binding site, and—optionally—end segments that promote integration of the expression cassette into a tumor genome (e.g., by homology directed repair).

When the composition is introduced into a subject, the gene editing system causes insertion of the nucleotide sequence encoding at least a segment of a cell surface protein into tumor-specific genomic material of the subject. The gene editing system may specifically target sequences exclusive to a tumor genome that have been identified via methods of the disclosure. For example, the tumor-specific genomic material may be detected by comparing tumor sequences to “matched normal” sequences, either of which may be obtained by next generation sequencing technologies. The methods may also include sequencing DNA obtained from the subject's sample.

In certain embodiments, the cell surface protein is an antigen and the Cas system includes a first ribonucleoprotein (RNP) that includes a Cas endonuclease and a guide RNA (gRNA). The composition may include a second RNP. By virtue of the gRNA, the RNP binds to a predetermined site in a tumor genome, cuts the tumor genome, and promotes integration of expression cassette there. The expression cassette includes an exogenous coding sequence. Once integrated into the tumor genome, the exogenous coding sequence is expressed as a cell surface protein (i.e., the antigen) on the surface of the tumor cell of a subject.

The composition may include a particle (e.g., lipid nanoparticle or liposome) containing the composition. I.e., the gene editing system, or nucleic acid encoding the gene editing system, and the expression cassette that includes the exogenous coding sequence may be enveloped or embedded in one or a plurality of delivery particles, such as liposomes or solid lipid nanoparticles that may include cationic lipids. The expression cassette may also include a promoter. For example, the composition may include at least: a first solid lipid nanoparticle comprising the expression cassette (for the cell surface protein) and a second solid lipid nanoparticle that includes at least one Cas endonuclease complexed with a guide RNA (gRNA) that targets the Cas endonuclease to a tumor genome. The particles may be provided at least one liposome enveloping one or more of the cell surface protein and the Cas endonuclease system.

Accordingly, aspects of the invention include compositions that specifically induce tumor cells to express a cell surface protein that provides an antigen to target those tumor cells for cell death. The cell surface protein is encoded as coding sequences in an expression cassette. The composition also include gene editing systems that exclusively edit tumor genomes to thereby induce tumor cells to express the coding sequences. After delivery of the composition, the target sequences in tumor genomes within tumor cells are recognized and cleaved by the gene editing system. Because healthy, non-tumor cells do not include those sequences in their genomes, the composition does nothing in those healthy, non-tumor cells. Only tumor cells are induced to express any antigen or antigenic peptide. The composition may contain the coding sequence for an exogenous antigen (i.e., cell surface protein) or an antigenic peptide (e.g., epitope) thereof. Preferably, the antigen or antigenic peptide is recognized by autologous T cells. By inducing expression of the antigen in tumor cells, the immune system responds to the presence of the antigen and begins to attack the tumor cells expressing the antigen, thereby treating cancer in the subject.

In some embodiments, methods and compositions of the disclosure use antibodies that bind to specifically to the exogenous cell surface proteins displayed only on tumor cells. Particularly, the invention provides compositions of antibodies specific to exogenous cell surface proteins displayed only on tumor cells that are conjugated to cytotoxic agents suitable for mediating killing of tumor cells.

Methods and compositions of the disclosure are useful for treating a patient affected by a cancer or proliferative disorder. Methods and compositions of the disclosure may be used for treatment of any cancer such as melanoma, leukemia, ovarian, breast, colorectal, or lung squamous cancer, sarcoma, renal cell carcinoma, pancreatic carcinomas, squamous tumors of the head and neck, brain cancer, liver cancer, prostate cancer, ovarian cancer, and cervical cancer.

### BRIEF DESCRIPTION OF THE DRAWINGS

FIG. 1 shows steps of a method for treating a tumor cell.

FIG. 2 diagrams a method for identifying tumor-specific genomic targets.

FIG. 3 shows a gene editing system specifically targeting tumor DNA.

FIG. 4 shows a CRISPR/Cas embodiment of a gene editing system.

FIG. 5 diagrams a method for treating cancer in a subject using a gene editing system.

### DETAILED DESCRIPTION

The disclosure provides methods and compositions that use a gene editing system for treatment of tumors. Compositions and methods of the disclosure are useful to induce expression of a cell surface protein (e.g., an antigen) exclusively on tumor cells and to use the presence of the exogenous antigen to mark the tumor cells for cell death. In some embodiments, the gene editing system include nucleases originally discovered in CRISPR systems.

Clustered regularly interspaced short palindromic repeats (CRISPR) were originally found in bacterial genomes under common control with various CRISPR-associated (Cas) proteins. Cas protein 9 (Cas9) has since proven to be an RNA-guided endonuclease useful as a gene editing system when complexed with guide RNA within a ribonucleoprotein (RNP). Cas9 is one Cas endonuclease and other, similar nucleases are known. Natively, the guide RNA included two short single-stranded RNAs, the CRISPR RNA (crRNA) that binds to the target in the target genetic material, and the trans-activating RNA (tracrRNA) that must also be present, although those two RNAs are commonly provided as a single, fused RNA sometimes called a single guide RNA (sgRNA). As used herein, guide RNA (gRNA) refers to either format. Cas9 and gRNA form a ribonucleoprotein (RNP) complex and bind to genomic DNA. The Cas9-gRNA complex scans the genome to identify a protospacer adjacent motif (PAM) and then a genomic DNA sequence adjacent to PAM that matches the gRNA sequence to cleave it. This scanning process depends on three-dimensional gRNA-dependent and gRNA-independent interactions of the Cas9-gRNA complex to DNA. The gRNA-dependent interaction is derived from the base-paring between a gRNA and genomic DNA. In contrast, the gRNA-independent interactions take place between genomic DNA and the amino acid residues of Cas9, including the PAM recognition. Thus, by virtue of the sequence of the gRNA, a Cas RNP cleaves target genetic material in a specific and controllable manner. Sequence-specific cleavage is useful for genome editing by, for example, providing a segment of DNA to be spliced in at the cleavage site by homology-directed repair.

To induce expression of a cell surface protein a CRISPR-associated (Cas) system can be delivered, along with an expression cassette for a cell surface protein, into a subject. The guide RNAs are designed and synthesized with predetermined targeting sequences and are thus unique reagents having a specific function. In Cas systems, the guide RNAs have sequences unique to a particular target site. The Cas system targets a predetermined site in a tumor genome and provides for the insertion of a coding sequence at that site in the tumor genome. The coding sequence preferably encodes a cell surface protein. Once the coding sequence is integrated at the predetermined site of the tumor genome (which may be, for example, a genomic safe harbor), the coding sequence, i.e., the cell surface protein, is then expressed in tumor cells. Because healthy, non-tumor cells do not have matching sites in their genomes, only the tumor cells then express the inserted cell surface protein, whereby the tumor cells can be destroyed. The tumor cells can be destroyed by a natural immune response, a primed immune response, or through the delivery of a cytotoxic antibody-drug-conjugate.

In certain vaccination or priming embodiments of the present disclosure, compositions that modulate the body's natural immune response function like vaccines in that the neoantigen itself may be separately provided such that it may prime the immune system. Such compositions preferably include the same antigen (i.e., cell surface protein) that is encoded by the coding sequence that is spliced specifically into tumor genomes. The increased presence of the antigen in the body causes an immune system response, whereby the immune system destroys only the cells (i.e., tumor cells) expressing the antigen.

In antibody drug conjugate (ADC) embodiments, a cytotoxic ADC targets the antigen that is expressed on the cell surface of the tumor cells. Such embodiments use a composition that includes an antibody conjugated to a cytotoxic drug (i.e., an ADC), in which the antibody is specific to the induced antigen. The antibody binds specifically to the antigen on the tumor cell's surface and the drug will destroy the tumor cell. Thus, the compositions and methods of the present invention are useful for treating cancer in a subject.

Methods and compositions of the invention are useful for treating any proliferative disease or disorder, such as cancer. The disclosure provides gene editing strategies, as well as methods and compositions that induce expression of a cell surface protein on a tumor-specific cell, or any cell in need of treatment, thereby addressing the lack of antigens recognizable by the immune system. Furthermore, the compositions and methods of the present invention can then use the presence of such antigens to their advantage by either attacking the cells head-on or by increasing the immune response.

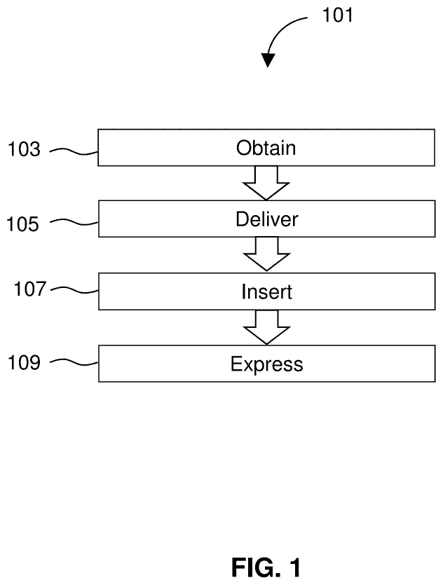

FIG. 1 diagrams a method 101 of inducing expression of a cell surface protein on a tumor cell using a gene editing system. In the method 101, a gene editing system is obtained 103 along with a coding sequence encoding at least a segment of a cell surface protein (e.g., an antigen) to be inserted into tumor-specific genomic material. The gene editing system and the nucleotide sequence encoding the cell surface protein are delivered 105 to a subject.

The gene editing system preferably includes a nuclease (i.e., a protein) such as a Cas endonuclease or a transcription activator like effector nuclease, or a nucleic acid that encodes the nuclease (such as a second expression cassette, plasmid, or other DNA segment for delivery). The nuclease preferably includes one or more nuclear localization signals (NLSs) to promote migration of the nuclease to the nucleus of tumor cells. Even when the nuclease is provided in a nucleic acid, e.g., in mRNA or DNA sense, it still may include the NLSs, in frame with the ORF for the nuclease. NLSs are short polypeptide sequences, e.g., about 10 to 25 amino acids long, and the sequences may be determined by searching literature, e.g., searching a medical library database for recent reports of nuclear localization signals.

The nucleotide sequence of the cell surface protein may be provided in or as an expression cassette. The expression cassette may include a promoter operably linked to the nucleotide sequence of the antigen. The expression of the nucleotide sequence in the expression cassette may be controlled by a constitutive promoter or of an inducible promoter that initiates transcription only when exposed to some particular external stimulus. The promoter can be linked to termination signals. Typically an expression cassette also includes sequences required for proper translation of the nucleotide sequence. For example, the expression cassette may include a sequence encoding an open reading frame (ORF) or segment thereof. The expression cassette may also comprise sequences not required for the expression of the nucleotide sequence. The expression cassette or the Cas system may include detectable labels to detect expression of the cell surface protein. The gene editing system then inserts 107 a nucleotide sequence encoding a cell surface protein into the tumor cells of the subject. The tumor cells then express 109 the cell surface protein on their cell surfaces.

Any antigen or antigenic peptide recognized by T cells or an ADC may be used in the present invention. Since tumor cells may suppress or mask the production of antigens, exogenous antigens can be used in the methods and compositions of the present invention. As such, in some embodiments of the invention, the expression cassette includes a nucleotide sequence encoding an exogenous antigen. The antigens may be synthetic antigens or peptides thereof. The expression cassette thus preferably includes an open reading frame (ORF) encoding the antigen. Any suitable sequence may be used as the nucleotide sequence of the ORF or the amino acid sequence of the antigen. For example, random, in-frame nucleotide sequences may be used or any amino acid sequence may be back-translated into nucleotide sequence using a codon chart or bioinformatics software. Software may be used to generate and test prospective neoantigens. For example, an arbitrarily large number (e.g., dozens or hundreds) of arbitrary (e.g., random) peptide sequences may be serially fed to a binding prediction software product, such as NetMCHpan 4.0 server to select a peptide sequence that will serve well as a neoantigen. See Jurtz, 2017, NetMHCpan-4.0: improved peptide-HMC Class I interaction predictions integrating eluted ligand and peptide binding affinity data, J Immunol 199(9):3360-3368, incorporated by reference. Those antigens may be used to stimulate a T cell or CTL response in vivo. In some embodiments, the antigen is an antigen that is not associated with cancer. In other embodiments, the antigen is an exogenous antigen that it is detectable by T cells as foreign.

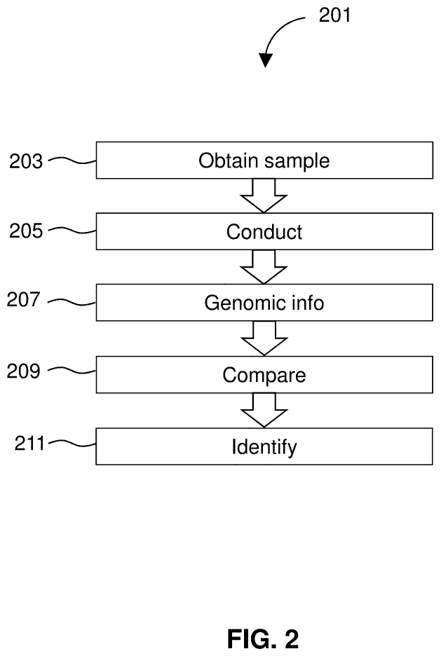

FIG. 2 diagrams a method 201 of identifying tumor-specific genomic material of a subject. In the method 201, a sample is obtained 203 from a subject. Patient samples are obtained 201 that preferably include both tumor DNA and healthy, non-tumor DNA. Samples may be obtained from any suitable germline or somatic sources (e.g., buccal or blood). Tumor cells may be obtained by tumor biopsy or circulating tumor cells may be isolated using methods known in the art.

An assay is conducted 205 on the sample and genomic information is obtained 207. For example, tumor and matched-normal DNA may be sequenced (e.g., on an Illumina sequencing instrument) to obtain tumor and matched-normal sequences. By such a manner, the genomic information of a non-tumor sample is compared 209 to genomic information of the tumor cell, and tumor-specific genomic material is identified 211 in the latter. For example, the whole-genome sequence of tumor and matched-normal DNA may be compared 209. Tumor-specific genomic material is identified 211 from the comparison. Comparing 209 may include comparing tumor sequences to matched-normal sequences (e.g., by alignment of assembled sequences from an NGS instrument run). Tumor-specific genomic material may include mutated genes specific to a tumor cell. Methods of the invention use the tumor-specific genomic material identified 211 by the method 201 as a target for Cas systems of the invention to cause insertion 107 of a nucleic acid sequence encoding a cell surface protein into such tumor-specific genomic material to express 109 the protein on the surface of the tumor cell.

Sequencing may be by any method known in the art. See, generally, Quail, et al., 2012, A tale of three next generation sequencing platforms: comparison of Ion Torrent, Pacific Biosciences and Illumina MiSeq sequencers, BMC Genomics 13:341. DNA sequencing techniques include classic dideoxy sequencing reactions (Sanger method) using labeled terminators or primers and gel separation in slab or capillary, sequencing by synthesis using reversibly terminated labeled nucleotides, pyrosequencing, 454 sequencing, Illumina/Solexa sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, polony sequencing, and SOLiD sequencing.

An example of a sequencing technology that can be used is Illumina sequencing. Illumina sequencing is based on the amplification of DNA on a solid surface using fold-back PCR and anchored primers. Genomic DNA is fragmented and attached to the surface of flow cell channels. Four fluorophore-labeled, reversibly terminating nucleotides are used to perform sequential sequencing. After nucleotide incorporation, a laser is used to excite the fluorophores, and an image is captured and the identity of the first base is recorded. Sequencing according to this technology is described in U.S. Pub. 2011/0009278, U.S. Pub. 2007/0114362, U.S. Pub. 2006/0024681, U.S. Pub. 2006/0292611, U.S. Pat. Nos. 7,960,120, 7,835,871, 7,232,656, 7,598,035, 6,306,597, 6,210,891, 6,828,100, 6,833,246, and 6,911,345, each incorporated by reference.

Another example of a DNA sequencing technique that can be used is the sequencing-by-ligation technology offered under the tradename SOLiD by Applied Biosystems from Life Technologies Corporation (Carlsbad, Calif.). In SOLiD sequencing, genomic DNA is sheared into fragments, and adaptors are attached to generate a fragment library. Clonal bead populations are prepared in microreactors containing beads, primers, template, and PCR components. Following PCR, the templates are denatured and enriched and the sequence is determined by a process that includes sequential hybridization and ligation of fluorescently labeled oligonucleotides.

Another example of a DNA sequencing technique that can be used is ion semiconductor sequencing using, for example, a system sold under the trademark ION TORRENT by Ion Torrent by Life Technologies (South San Francisco, Calif.). Ion semiconductor sequencing is described, for example, in Rothberg, et al., An integrated semiconductor device enabling non-optical genome sequencing, Nature 475:348-352 (2011); U.S. Pubs. 2009/0026082, 2009/0127589, 2010/0035252, 2010/0137143, 2010/0188073, 2010/0197507, 2010/0282617, 2010/0300559, 2010/0300895, 2010/0301398, and 2010/0304982, each incorporated by reference. DNA is fragmented and given amplification and sequencing adapter oligos. The fragments can be attached to a surface. Addition of one or more nucleotides releases a proton (H+), which signal is detected and recorded in a sequencing instrument.

Other examples of a sequencing technology that can be used include the single molecule, real-time (SMRT) technology of Pacific Biosciences (Menlo Park, Calif.) and nanopore sequencing as described in Soni and Meller, 2007 Clin Chem 53:1996-2001. Such sequencing methods are useful when obtaining large fragments of DNA from a reference or test sample, such as in the methods described in U.S. Pub. 2018/0355408, the contents of which are incorporated by reference herein.

Sequencing tumor DNA provides tumor sequences that may be analyzed to identify tumor-specific DNA sequences that appear exclusively in tumor genomes and do not appear in the a genome from a healthy, non-tumor cell from the same subject.

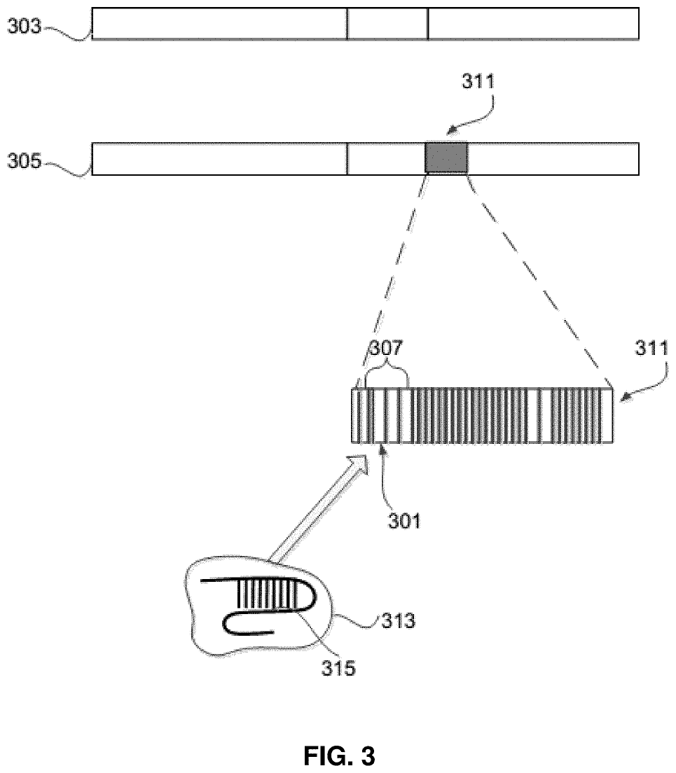

FIG. 3 illustrates the analysis of tumor sequence 305 to identify tumor-specific genomic material 311. In the depicted embodiment, tumor sequence 305 is aligned to matched normal sequences 303 to determine any differences. Where the tumor sequences 305 include tumor-specific genomic material 311 that are not also present in the matched normal sequences 303, that tumor-specific genomic material 311 provides a target for cleavage by a gene editing system and subsequent integration (e.g., by homology directed repair) of an expression cassette bearing, e.g., exogenous coding sequence.

More particularly, in the depicted embodiment, a segment 307 of the tumor-specific genomic material 311 (e.g., DNA) is shown. The gene editing system is designed to recognize that segment and cleave the tumor DNA at a target 301. Because the matched normal DNA does not include the tumor-specific genomic material 311, a healthy, non-tumor genome does not include a corresponding segment 307 that can be recognized by the gene editing system 313 and thus the gene editing system 313 has no relevant effect on healthy, non-tumor cells. A distinguishing feature of the segment 307 is that the segment 307 includes features that satisfy the targeting requirement of the gene editing system 313. Thus, a distinguishing feature of the tumor-specific material 311 is that it is not also found in “matched normal” sequences from healthy, non-tumor cells. The segment 307 within the tumor material 311 includes matches for the targeting sequence of gene editing system 313. Where, for example, the gene editing system 313 uses a Cas endonuclease, the segments 307 are those locations that include a suitable PAM adjacent to a suitably specific approximately 20 base target.

Using this information, one of skill in the art can prepare or obtain gene editing systems useful to insert a copy of a nucleotide sequence encoding a cell surface protein at the target 301. For example, one may access the sequence of the tumor-specific genomic material from the method 201 of comparing 209 germline DNA to tumor DNA to search for and identify targets suitable for insertion and editing with a gene editing system 313.

In a preferred embodiment, the gene editing system uses Cas endonuclease and guide RNA. For example, the Cas endonuclease may be Cas9 from Streptococcus pyogenes (spCas9). The Cas endonuclease may be complexed with a guide RNA 315 as a ribonucleoprotein (RNP). One of skill in the art may design the gRNA 315 to have a 20-base targeting sequence complementary to the segment 307 of the tumor-specific genomic material 311. Alternatively, the gRNA 315 may have a 20-base targeting sequence complementary to a target within a few hundred or thousand bases of the segment 307.

The target may be a sequence describable as 5′-20 bases-protospacer adjacent motif (PAM)-3′, where the PAM depends on Cas endonuclease (e.g., NGG for Cas9). To insert an exogenous cell surface protein, two Cas RNPs may be used along with a pair of guide RNAs 309 to flank the target 301. The RNPs bind to their cognate targets in the tumor-specific DNA 305 and introduce double stranded breaks. The exogenous cell surface protein being inserted may have ends that are homologous to sequences flanking the target 301 to induce the cell's endogenous homology-directed repair response, to repair the genome by inserting the exogenous DNA segment. See How, 2019, Inserting DNA with CRISPR, Science 365(6448):25 and Strecker, 2019, RNA-guided DNA insertion with CRISPR-associated transposases, Science 365(6448):48, both incorporated herein by reference. Thus, in the depicted embodiment, the sequence encoding the cell surface protein is inserted into the tumor-specific DNA 311 only using a CRISPR/Cas nuclease system. The method 101, may be performed with any suitable gene editing system. A Cas nuclease system uniquely corresponds to intended targets, such as a predetermined site in the tumor-specific genomic material. The predetermined site may be near the promoter region of a tumor specific gene. In some embodiments, the target site may be within an open reading frame (ORF) in the tumor-specific genomic material, and genome editing can integrate the exogenous coding sequence, in-frame, within the ORF. Insertion of the coding sequence into the ORF causes expression of the antigen on the cell surface. Gene editing systems can be designed and synthesized or ordered by making reference to the predetermined site in the tumor-specific genomic material. Alternatively, nucleotide sequence of a cell surface protein (e.g., an antigen) and a suitable promoter can be expressed in a safe harbor, using Cas systems described herein.

Embodiments of the invention use any suitable gene editing system such as, for example, CRISPR systems, transcription activator like effector nucleases (TALENs), zinc finger nucleases, or meganucleases. In any embodiment discussed herein, gene editing system may be taken to refer to compositions that include an active form of the protein or that include a nucleic acid encoding the gene editing system. Thus, a CRISPR system can include a Cas-endonuclease complexed with a guide RNA as an RNP, or a nucleic acid encoding those elements, such as on a plasmid or other expression cassette. Preferred embodiments of the invention use a CRISPR-associated (Cas) endonuclease. The gene editing system includes a protein (i.e., a Cas endonuclease) that is complexed with target-specific gRNA, thus forming a complex that targets the Cas endonuclease to a specific sequence in the tumor-specific genomic material. Any suitable Cas endonuclease or homolog thereof may be used. A Cas endonuclease may be Cas9 (e.g., spCas9), Cpf1 (aka Cas12a), C2c2, Cas13, Cas13a, Cas13b, e.g., PsmCas13b, LbaCas13a, LwaCas13a, AsCas12a, PfAgo, NgAgo, CasX, CasY, others, modified variants thereof, and similar proteins or macromolecular complexes.

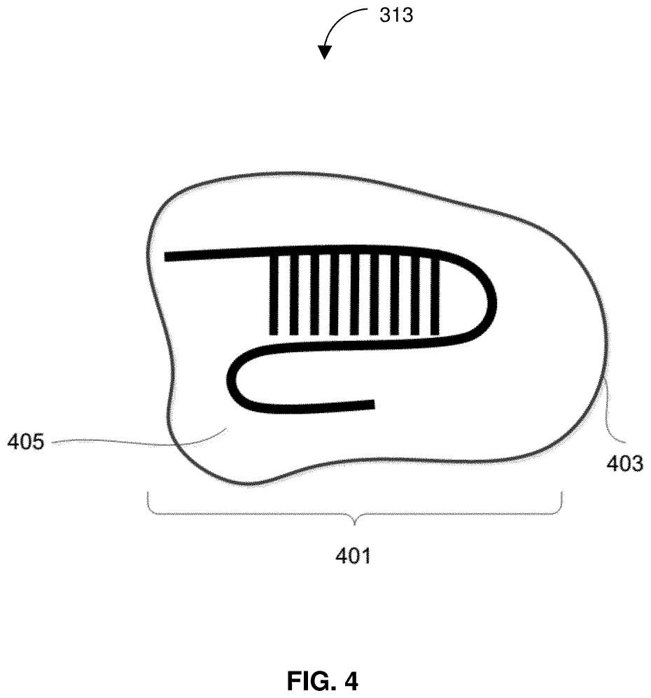

FIG. 4 shows an embodiment of a gene editing system 313. The depicted embodiment includes a Cas endonuclease 403 and a guide RNA 405 (i.e., gRNA). The gRNA 405 includes a targeting sequence of approximately 20 bases complementary or nearly complementary to a target in tumor-specific genomic material of a subject. The Cas endonuclease 403 and gRNA 405 are complexed together into a ribonucleoprotein (RNP) 401. The CRISPR/Cas system 313 in a composition or method of the disclosure may include at least one Cas endonuclease 403 (or a nucleic acid encoding the Cas endonuclease).

The host bacteria capture small DNA fragments (˜20 bp) from invading viruses and insert those sequences (protospacers) into their own genome to form a CRISPR. CRISPR regions are transcribed as pre-CRISPR RNA (pre-crRNA) and processed to give rise to target-specific crRNA. Invariable target-independent trans-activating crRNA (tracrRNA) is also transcribed from the locus and contributes to the processing of precrRNA. The crRNA and tracrRNA have been shown to be combinable into a single guide RNA (gRNA). As used herein, “guide RNA” or gRNA refers to either format. The gRNA forms a RNP with Cas9, and the RNP cleaves a target that includes a portion complementary to the guide sequence in the gRNA, as well as a sequence known as protospacer adjacent motif (PAM). The RNPs are programmed to target a specific viral nucleic acid by providing a gRNA having a ˜20-bp guide sequence that is complementary or substantially complementary to a target in viral nucleic acid. The targetable sequences include, but are not limited to: 5″-X 20NGG-3′ or 5′-X 20NAG-3′; where X 20 corresponds to the 20-bp crRNA sequence and NGG and NAG are PAMs. Sequences with lengths other than 20 bp and PAMs other than NGG and NAG are known and are included within the scope of the invention.

Any of the CRISPR/Cas system compositions and methods of the disclosure may be included in any suitable format, and including any of protein, messenger RNA, DNA, RNP, or a combination thereof. For example, delivery of RNPs into cells may be by electroporation, chemical poration, or via liposomal mediated delivery. The nucleotide sequence encoding a cell surface protein may be included as a segment of DNA that also includes one or more of a promoter, a fluorescent protein, an SV40 sequence, and a poly(A) sequence. The nucleotide sequence encoding a cell surface protein may be included in an expression cassette along with one or more of a promoter, a fluorescent protein, an SV40 sequence, and a poly(A) sequence. The sequence (e.g., expression cassette) and/or the gene editing system may be delivered as a plasmid or other similar vector. The components of the systems may be delivered in a DNA-sense (e.g., as a plasmid or in a viral vector) for transcription and translation into active proteins in the tumor cells. In some embodiments, a gene editing system 313 is delivered as nucleic acid, e.g., the Cas endonuclease, and is packaged with a nucleotide sequence encoding a cell surface protein using one or more lentiviral or adeno-associated virus (AAV) vector.

The gene editing system may be delivered in a protein, RNP, DNA, or mRNA format dependent on a desired persistence or stability in the tumor cells. The gene editing system may include an endonuclease designed to introduce a cell surface protein into a target site of the tumor specific genomic material. Preferred target sites may include a gene locus of a tumor cell gene, a predetermined site in tumor-specific genomic material, such as a tumor-specific locus of a tumor-specific gene of a subject or a genomic safe harbor (e.g., a safe harbor such as AAVS1, CCR5, or ROSA26.). The gene editing system may be included as DNA that is transcribed after the composition is introduced into subject as mRNA or as a protein or RNP. Regardless of format, a suitable packaging vector or particle may be used.

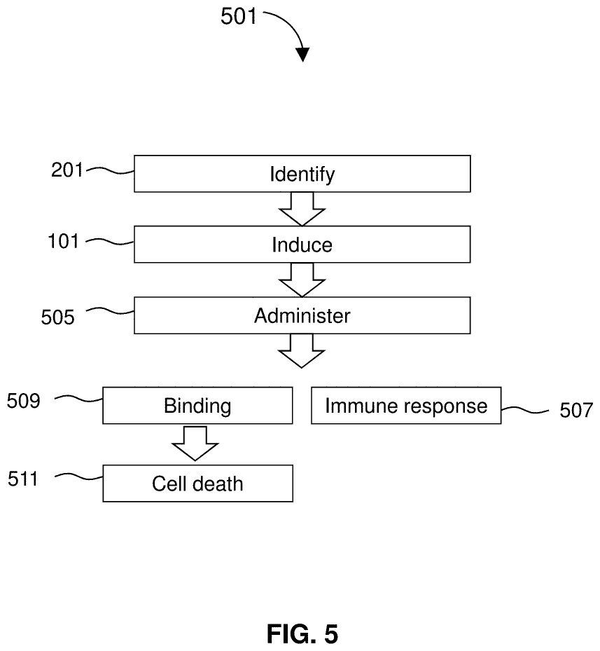

FIG. 5 diagrams an exemplary method 501 for treating cancer in a subject using the gene editing system 313 of the present invention. In the method 501, the method of identifying tumor-specific genomic material of a subject 201 is performed. Upon identification of tumor-specific genomic material of the subject, the method 101 is performed. Once the tumor cells express 109 the cell surface protein on their cell surface, a composition is administered 505 to the subject. The composition is specific to the cell surface protein expressed 109 on the tumor cell surface.

In one embodiment, the composition may include the same cell surface protein expressed on the tumor cell. An immune system response is induced 507 upon administering 505 a composition that includes the same cell surface protein expressed 109 on the tumor cell. Compositions comprising the same cell surface protein are described hereinafter.

In another embodiment, an antibody-drug conjugate (ADC) is delivered, in which the antibody binds to to the cell surface protein, and is conjugated to a cytotoxic drug. The antibody specifically binds 509 to the cell surface protein upon administering 505 the compositions. A biochemical reaction between the antibody and the target cell surface protein triggers a signal in the tumor cell. The tumor cell absorbs or internalizes the antibody together with the linked drug. After the ADC is internalized, the tumor cell is destroyed 511 (killed) by the cytotoxic agent. Such targeting limits side effects and gives a wider therapeutic window than other chemotherapeutic agents. The therapeutic antigen-specific compositions described above are described in detail hereinafter.

Methods of the invention also include inhibiting tumor growth or metastasis of cancer in a subject by administering to the subject a therapeutically effective amount of the compositions disclosed herein. A therapeutically effective amount of the compositions disclosed herein is an amount sufficient to inhibit growth, replication or metastasis of cancer cells, or to inhibit a sign or a symptom of the cancer. The therapeutically effective amount may depend on disease severity, the type of disease, or the subject's general health.

Any suitable delivery system may be used to deliver gene editing systems of the present invention. Delivery methods are described in detail in Wilbie, 2019, Delivery aspects of CRISPR/Cas for in vivo genome editing, Acc Chem Res 18; 52(6):1555-1564, incorporated by reference. Preferably, non-viral delivery of the gene editing systems of the present invention are used. For example, liposome(s) may be used to deliver a gene editing system or nucleic acid encoding the gene editing system along with an expression cassette for an exogenous coding sequence. Any nucleic acid delivered may be as a plasmid that may also include a segment that encodes a gRNA. Where the liposome packages nucleic acids, the nucleic acids may include one or any combination of a plasmid, a guide RNA, and the expression cassette. Compositions may be packaged in a plurality of the liposomes. Each of the plurality of liposomes may envelope one or more of an expression cassette and/or the gene editing system (e.g., in protein or plasmid format). Delivery of the liposomes to tumor cells in a subject causes those cells to express the antigen in a stable manner.

Other embodiments use lipid nanoparticles such as solid lipid nanoparticles. A lipid nanoparticle (LNP) may include a gene editing system. LNPs may be about 100-200 nm in size and may optionally include a surface coating of a neutral polymer such as PEG to minimize protein binding and unwanted uptake. The nanoparticles are optionally carried by a carrier, such as water, an aqueous solution, suspension, or a gel. For example, LNPs may be included in a formulation that may include chemical enhancers, such as fatty acids, surfactants, esters, alcohols, polyalcohols, pyrrolidones, amines, amides, sulfoxides, terpenes, alkanes and phospholipids. LNPs may be suspended in a buffer. The buffer may include a penetration enhancing agent such as sodium lauryl sulfate (SLS). SLS is an anionic surfactant that enhances penetration into the skin by increasing the fluidity of epidermal lipids. Lipid nanoparticles may be delivered via a gel, such as a polyoxyethylene-polyoxypropylene block copolymer gel (optionally with SLS). Poloxamers are nonionic triblock copolymers composed of a central hydrophobic chain of polyoxypropylene (poly(propylene oxide)) flanked by two hydrophilic chains of polyoxyethylene (poly(ethylene oxide)). Because the lengths of the polymer blocks can be customized, many different poloxamers exist having different properties. For the generic term “poloxamer”, these copolymers are commonly named with the letter “P” (for poloxamer) followed by three digits: the first two digits×100 give the approximate molecular mass of the polyoxypropylene core, and the last digit×10 gives the percentage polyoxyethylene content (e.g. P407=poloxamer with a polyoxypropylene molecular mass of 4,000 g/mol and a 70% polyoxyethylene content). LNPs may be freeze-dried (e.g., using dextrose (5% w/v) as a lyoprotectant), held in an aqueous suspension or in an emulsification, e.g., with lecithin, or encapsulated in LNPs using a self-assembly process. LNPs are prepared using ionizable lipid L319, distearoylphosphatidylcholine (DSPC), cholesterol and PEG-DMG at a molar ratio of 55:10:32.5:2.5 (L319:DSPC:cholesterol:PEG-DMG). The payload may be introduced at a total lipid to payload weight ratio of ˜10:1. A spontaneous vesicle formation process is used to prepare the LNPs. Payload is diluted to ˜1 mg/ml in 10 mmol/l citrate buffer, pH 4. The lipids are solubilized and mixed in the appropriate ratios in ethanol. Payload-LNP formulations may be stored at ˜80° C. See Maier, 2013, Biodegradable lipids enabling rapidly eliminating lipid nanoparticles for systemic delivery of RNAi therapeutics, Mol Ther 21(8):1570-1578, incorporated by reference. See, WO 2016/089433 A1, incorporated by reference herein.

Compositions of the disclosure may include a plurality of lipid nanoparticles having the cell surface protein and the gene editing system embedded therein. In one embodiment, a plurality of lipid nanoparticles comprises at least a solid lipid nanoparticle comprising a segment of DNA that encodes the cell surface protein; a second solid lipid nanoparticle that includes at least one Cas endonuclease complexed with a gRNA that targets the CRISPR/Cas system to a locus within a predetermined site in tumor-specific genomic material of a subject.

Another embodiment of the present invention is directed to a composition comprising a cell surface protein or portion thereof, such as an antigen or an antigenic peptide (e.g., epitope). Preferably, the antigen or antigenic peptide is recognized by T cells. Any antigen may be used in the present invention that is displayed or detected on the surface of tumor cells. Preferably, the antigen is the same antigen expressed on tumor cells by methods e.g., 101 of the present invention. Such antigens are exogenous antigens that are recognized as “foreign” or “non-self” by the immune system.

Any antigen or antigenic peptide recognized by T cells may be used in the present invention. Since tumor cells suppress or mask the production of antigens, exogenous antigens can be used in the methods and compositions of the present invention. The antigens correspond to the amino acid sequence of the antigen expressed on the cell via methods e.g., 101 of the present invention. The antigens may be synthetic antigens or peptides thereof. These antigens can be used to stimulate a T cell or CTL response in vivo. In some embodiments, the antigen is an antigen that is not associated with cancer. In other embodiments, the antigen is an exogenous antigen that it is detectable by T cells as foreign.

In some embodiments, the antigen is an antigen that is not associated with cancer. The antigen can be an exogenous antigen so that it is detectable by T cells as foreign. The antigen may be a synthetic antigen. Thus, methods of the invention include inducing the expression of the antigen on a tumor cell that is not associated with cancer or tumor growth, and is thus detectable by T cells when tumor cells are present.

Accordingly, compositions of the present invention include an antigen present in the form of a nucleotide sequence encoding the antigen. The antigen is the same antigen expressed on tumor cells by methods of the present invention. As such, the nucleotide sequence encoding the antigen may be present on a vector that is delivered to a subject. In particular, one or more antigenic peptides may be delivered in the composition. In some embodiments, the composition may also include and immune checkpoint antagonist. Methods of preparing antigens are known in the art. See, Hos, 2018, Approaches to improve chemically defined synthetic peptide vaccines, Front Immunol 9:884, incorporated by reference. One of skill in the art can readily prepare (or obtain) a synthetic antigen using such methods for use in the disclosed composition for treating cancer.

Certain embodiments use antibody-drug conjugates (ADCs). The antibody is specific to the antigen expressed on the surface of the tumor cells by methods of the present invention. Typically, the drug to which the antibody is conjugated to is a cytotoxic agent. The drug may be more potent than those used for traditional cancer treatments as the ADCs of the present invention are capable of specifically targeting patient-specific tumor cells. Exemplary cytotoxic agents include drugs, enzymes, cytokines, radionuclides, photodynamic agents and molecules that induce apoptosis of a tumor cell. For example, such agents may include auristatins, fludarabine, chlorambucil, daunorubicin, doxorubicin (e.g., in liposomes), an indolino-benzodiazepine dimer, a puromycin, a tubulysin, a hemiasterlin, a spliceostatin, a pladienolide, stereoisomers, isosteres cisplatin, bleomycin, maytansinoids melphalan, mitomycin-C, and methotrexats, pyrrolobenzodiazepines (PDBs) calicheamicin, nemorubicin, PNU-159682, anthracycline, vinca alkaloid, taxane, trichothecene, CC1065, camptothecin, elinafide, a combretastain, a dolastatin, a duocarmycin, an enediyne, a geldanamycin, analogs, and derivatives thereof that have cytotoxic activity. ADCs typically comprise a 1:2 to 1:4 ratio of antibody to drug.

The antibody and drug can be linked by a cleavable linker, or non-cleavable linker. In a preferred embodiment, the linker is a non-cleavable linker so that systemic release of the cytotoxic drug is prevented, reducing or eliminating off-target toxicity. As such, release of the cytotoxic agent does not occur before the ADC is internalized by the tumor cell when using a non-cleavable linker. Upon entering the lysosome of the tumor cell, the antibody is digested by lysosomal proteases, resulting in the release of the cytotoxic agent, and thus the destruction of the tumor cell expressing the antigen.

Any method known in the art of conjugating a drug to an antibody may be used to produce site-specific ADCs of the present invention. For example, glycan engineering may be used to conjugate the antibody to the drug. See Qasba, 2008, Site-specific linking of biomolecules via glycan residues using glycosyltransferases, Biotechnol Prog 24(3):520-6; U.S. Pub. 2007/0258986; and U.S. Pub. 2006/0084162, all incorporated by reference.

The antibodies described herein may be natural monoclonal antibodies or synthetic antibodies, such as recombinant antibodies, non-immunoglobulin derived synthetic antibodies, or affimer proteins. Exemplary antibodies include antibodies having affinity and selectivity for cell surface proteins induced by the methods of the invention. Exemplary antibodies include: anti-p53 antibody, anti-HER-2/neu antibody, anti-EGFR antibody, anti-cathepsin D antibody, anti-Bcl-2 antibody, anti-E-cadherin antibody, anti-CA125 antibody, anti-CA15-3 antibody, anti-CA19-9 antibody, anti-c-erbB-2 antibody, anti-P-glycoprotein antibody, anti-CEA antibody, anti-retinoblastoma protein antibody, anti-ras oncoprotein antibody, anti-Lewis X antibody, anti-Ki-67 antibody, anti-PCNA antibody, anti-CD3 antibody, anti-CD4 antibody, anti-CD5 antibody, anti-CD7 antibody, anti-CD8 antibody, anti-CD9/p24 antibody, anti-CD10 antibody, anti-CD11c antibody, anti-CD13 antibody, anti-CD14 antibody, anti-CD15 antibody, anti-CD19 antibody, anti-CD20 antibody, anti-CD22 antibody, anti-CD23 antibody, anti-CD30 antibody, anti-CD31 antibody, anti-CD33 antibody, anti-CD34 antibody, anti-CD35 antibody, anti-CD38 antibody, anti-CD41 antibody, anti-LCA/CD45 antibody, anti-CD45RO antibody, anti-CD45RA antibody, anti-CD39 antibody, anti-CD100 antibody, anti-CD95/Fas antibody, anti-CD99 antibody, anti-CD106 antibody, anti-ubiquitin antibody, anti-CD71 antibody, anti-c-myc antibody, anti-cytokeratins antibody, anti-vimentins antibody, anti-HPV proteins antibody, anti-kappa light chains antibody, anti-lambda light chains antibody, anti-melanosomes antibody, anti-prostate specific antigen antibody, anti-S-100 antibody, anti-tau antigen antibody, anti-fibrin antibody, anti-keratins antibody and anti-Tn-antigen antibody.

Methods of making monoclonal antibodies are known in the art and described in, for example, Antibodies: A Laboratory Manual, Second edition, edited by Greenfield, Cold Spring Harbor Laboratory Press (2014) ISBN 978-1-936113-81-1. Methods of making synthetic antibodies are described in, for example, US 2014/0221253; US 2016/0237142; and Miersch, 2012, Synthetic antibodies: concepts, potential and practical considerations, Methods 57(4):486-98, all incorporated by reference.

The ADCs provided herein include a drug (such as a cytotoxic agent) conjugated to a monoclonal antibody that specifically binds to the antigen expressed on the cell surface of the tumor cells by the methods of the invention. An ADC may optionally include a linker. For example, the linker can be a bifunctional or multifunctional moiety that links one or more drug moieties to an antibody to form an ADC. The linker, having reactive properties, covalently attaches to the drug and to the antibody or a cysteine thiol of an antibody forms a bond with a functional group of a linker thereby forming an ADC. Any linker with a reactive function may be used. A linker is capable of reacting with an electrophilic group present on an antibody. Such linkers include, but are not limited to, maleimide, haloacetamides, oc-haloacetyl, activated esters (e.g., succinimide esters, 4-nitrophenyl esters, pentafluorophenyl esters, tetrafluorophenyl esters), anhydrides, acid chlorides, sulfonyl chlorides, isocyanates, and isothiocyanates. In some embodiments, the linker is a cleavable linker and facilitates the release of the drug. Such cleavable linkers include acid-labile linkers, protease-sensitive linkers, photolabile linkers, and disulfidecontaining linkers (Chari et al, Cancer Res 52:127-131, 1992; U.S. Pat. No. 5,208,020, both incorporated by reference). The ADCs disclosed herein can be used for the treatment of any type of cancer alone or in combination with another drug and can be used in combination with any standard therapy for the treatment of cancer.

In methods of treating cancer according to the disclosure, a therapeutically effective amount of a composition is administered to a subject. A therapeutic amount is an amount that is sufficient to cause a cancer cell to express an exogenous cell surface protein as an antigen that marks the cell for cell death. Accordingly, methods of the disclosure include treating cancer in a subject by administering to the subject a therapeutically effective amount of the compositions disclosed herein.

In general, an effective dosage of any of the compositions of the present invention can readily be determined by a skilled person, having regard to typical factors such as the age, weight, sex and clinical history of the patient. A typical dosage could be, for example, 1-1,000 mg/kg, preferably 5-500 mg/kg per day, or less than about 5 mg/kg, for example administered once per day, every other day, every few days, once a week, once every two weeks, or once a month, or a limited number of times, such as just once, twice or three or more times. Methods of the invention include delivering an effective amount of the composition to the subject such that expression of the antigen is induced on tumor cell surfaces and then either of the compositions for treating cancer disclosed herein is delivered to the same subject in therapeutically effective amounts.

The disclosure also provides pharmaceutical compositions of the compositions described herein. Compositions may be formulated for delivery by any route of administration. For example, compositions may be formulated for oral, enteral, parenteral, subcutaneous, intravenous, or intramuscular administration.

Formulations may provide aqueous suspensions, oil suspensions, dispersible powders, or emulsions. The aqueous suspensions may contain one or more compounds in admixture with excipients suitable for the manufacture of aqueous suspensions. Oily suspensions may be formulated by suspending the compound in a suitable oil such as mineral oil, arachis oil, olive oil, or liquid paraffin. The oily suspensions may contain a thickening agent, for example beeswax, hard paraffin or cetyl alcohol. Dispersible powders and granules suitable for preparation of an aqueous suspension by the addition of water provide the compound in admixture with a dispersing or wetting agent, suspending agent and one or more preservatives. Suitable dispersing or wetting agents and suspending agents are exemplified, for example sweetening, flavoring and coloring agents, may also be present.

The compositions may also be in the form of oil-in-water emulsions. The oily phase may be a lipid, a mineral oil, for example liquid paraffin or mixtures of these. Suitable emulsifying agents may be naturally-occurring gums, for example gum acacia or gum tragacanth, naturally occurring phosphatides, for example soya bean, lecithin, and esters or partial esters derived from fatty acids and hexitol anhydrides, for example sorbitan monooleate and condensation products of the said partial esters with ethylene oxide, for example polyoxyethylene sorbitan monooleate.

Compositions may include other pharmaceutically acceptable carriers, such as sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin (glycerol), erythritol, xylitol. sorbitol, mannitol and polyethylene glycol; esters, such asethyl oleate and ethyllaurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Compositions may be in a form suitable for oral use. For example, oral formulations may include tablets, troches, lozenges, fast-melts, aqueous or oily suspensions, dispersible powders or granules, emulsions, hard or soft capsules, syrups or elixirs. Formulations for oral use may also be presented as hard gelatin capsules in which the citrate, citric acid, or a prodrug, analog, or derivative of citrate or citric acid is mixed with an inert solid diluent, for example calcium carbonate, calcium phosphate or kaolin, or as soft gelatin capsules in which the compound is mixed with water or an oil medium, for example peanut oil, liquid paraffin or olive oil.

Pharmaceutical compositions of the disclosure may be in the form of a sterile injectable aqueous or oleaginous suspension. This suspension may be formulated according to the known art using those suitable dispersing or wetting agents and suspending agents which have been mentioned above. The sterile injectable preparation may also be in a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid find use in the preparation of injectables.

Any of the compositions may be included in a kit. The kit may include components of a gene editing system, an expression vector that includes a coding sequence, and additional reagents and instructions that promote integration of the coding sequence into a tumor genome. The additional reagents may include one or more of a polymerase, a ligase, dNTPs, a co-factor, and a topoisomerase. The kit may include one or more tools for delivering the expression cassette and the gene editing system into a subject. For example, the kit may include a syringe or other surgical tool for delivering the composition to the subject. Optionally, the expression cassette may include a promoter or a transcription factor binding site to increase transcription of the antigen. The kit or the composition may be used in a method of inducing expression of a cell surface protein on a tumor cell of a subject.

The kit may also include compositions for treating cancer in the subject. The cancer treatment compositions may include those described herein that are specific to the antigen being expressed on tumor cells by methods of the present invention. Alternatively, the kit or the composition may be used in conjunction with other kits or compositions of the present invention to treat cancer in the subject.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. A method of treating a tumor cell, the method comprising:
introducing, into a subject, a gene editing system in the form of a ribonucleoprotein (RNP) comprising Cas endonuclease complexed with guide RNA, the guide RNA comprising a targeting sequence complementary to a target in tumor DNA from a subject that is not found in matched normal sequences from non-tumor cells of the subject and an expression cassette including a coding sequence encoding at least a segment of a cell surface protein,
wherein the gene editing system integrates the expression cassette into a genome of a tumor cell in the subject, thereby causing the tumor cell to express the coding sequence as a neoantigen.

2. The method of claim 1, wherein the neoantigen marks the tumor cell for destruction by an immune response of the subject or an antibody-drug-conjugate.

3. The method of claim 1, wherein the expression cassette comprises end segments that promote integration of the expression cassette into a tumor genome by homology directed repair.

4. The method of claim 3, wherein the Cas endonuclease comprises at least one nuclear localization signal (NLS).

5. The method of claim 1, wherein the method includes delivering the neoantigen to the subject prior to the introducing step to thereby prime an immune system of the subject.

6. The method of claim 1, wherein the method includes, prior to the introducing step, obtaining tumor DNA from the subject and analyzing the tumor DNA to identify a target in the tumor DNA that is not found in matched normal sequences from healthy, non-tumor cells of the subject.

7. The method of claim 6, wherein the analyzing step includes sequencing tumor DNA.

8. The method of claim 7, further comprising:
sequencing matched, normal DNA from the healthy, non-tumor cells of the subject to thereby obtain tumor sequences and matched normal sequences;
aligning the tumor sequences to the matched normal sequences; and
identifying the target as a section of the tumor sequence that does not have an exact match in the matched normal sequences.

9. The method of claim 6, further comprising synthesizing one or more guide RNAs with targeting portions that are complementary to the target in the tumor DNA when the target in the tumor DNA is adjacent a protospacer adjacent motif in the tumor DNA.

10. The method of claim 1, wherein said expression cassette further comprises a promoter operably linked to the coding sequence.

11. The method of claim 1, wherein the neoantigen is recognized by a receptor on a T cell in the subject.

12. The method of claim 1, further comprising administering, to the subject, an antibody-drug-conjugate (ADC) comprising an antibody that specifically binds the neoantigen.

13. The method of claim 12, wherein the ADC includes the antibody conjugated to a cytotoxic drug that kills the tumor cell.

14. The method of claim 1, further comprising:
analyzing a sample from the subject to identify a target in and specific to the genome of the tumor cell in the subject;
obtaining guide RNA that hybridizes to the target;
introducing the guide RNA to the Cas endonuclease that includes a nuclear localization signal to form the ribonucleoprotein (RNP); and
packaging the RNP and the expression cassette in one more lipid nanoparticles for delivery.

15-20. (canceled)
